(19) United States
(12) Patent Application Publication
(10) Pub. No.: US 2024/0269096 A1
(43) Pub. Date: Aug. 15, 2024

(54) Rapid Onset Therapeutic Ketosis Enabled by D-BetaHydroxyButyric Acid Systems, Compounds and Methods for Sepsis Protection and Enhanced Medical Recovery

(71) Applicant: American Ketone Therapeutics LLC, Sheridan, WY (US)

(72) Inventor: Heidi H. Hynes, Seattle, WA (US)

(21) Appl. No.: 18/401,445

(22) Filed: Dec. 30, 2023

Related U.S. Application Data
(60) Provisional application No. 63/478,142, filed on Dec. 31, 2022.

(51) Int. Cl. A61K 31/19; A61B 5/00; A61B 5/145; A61K 45/06

(52) U.S. Cl. CPC A61K 31/19; A61B 5/14532; A61B 5/14546; A61B 5/412; A61K 45/06

(57) ABSTRACT

The claimed uses of bioidentical free D-BetaHydroxyButyric Acid as a method (1303), system (1411) and optional co-adjuvant compound for pharmaceutical co-administration with sepsis therapeutic pharmaceutical compounds to treat sepsis and improve hospital recovery are novel, safe and not anticipated by the body of research spanning well over 75 years. Overall, no adverse findings have been reported within cell, animal and human safety studies at high dose levels with D-BetaHydroxyButyric acid which supports the safety and efficacy of the claimed uses at the anticipated usage levels for the disclosed applications.

### CROSS-REFERENCE TO RELATED APPLICATIONS

This patent application is related to and claims priority from previously filed provisional application Ser. No. 63/478,142 filed on Dec. 31, 2022.

### TECHNICAL FIELD

The claimed invention has applicability in hospital recovery medication. With greater particularity, the claimed invention is relevant to therapeutic interventions for sepsis. With still greater particularity, the claimed invention is directed at novel uses of the ketone body D-BetaHydroxyButyric Acid for treatment of sepsis together with and independently of pharmaceutical sepsis medication with related systems and methods.

### BACKGROUND OF THE INVENTION

Latest research published in The Lancet has found that sepsis is responsible for the most deaths worldwide, even more than cancer or coronary disease previously believed to be the leading causes of death globally. Sepsis occurs when chemicals are released in the bloodstream to fight an infection triggering inflammation throughout the body. This can cause a cascade of changes that damage multiple organ systems, leading them to fail, sometimes even resulting in death. While treatment includes antibiotics and intravenous fluids, these often treat the symptoms and do not address or mitigate the subsequent inflammation from sepsis. Individuals undergoing a post-surgical hospital stay are at particular risk of contracting sepsis.

Cardiac bypass is the most common type of heart surgery performed on adults in the US. The average cost of bypass surgery in the US in 2018 was $123,000. With an average hospital stay of 6-7 days for bypass surgery, each and every day of shortened hospital stay not only improves recovery outcomes but also reduces the likelihood of return visits due to complications. With respect to cancer surgery hospitalizations, room for improvement exists over current therapeutic approaches.

### BRIEF SUMMARY OF THE INVENTION

Experimental evidence suggests ketosis could improve many of the coexisting pathophysiological processes sepsis patients have such as: inflammation, muscle mass loss, hyperglycemia, decreased myocardial contractility and some pathogen specific immune response pathways like those against SARS-Cov-2 and Pseudomonas. Ketosis may be naturally induced through strict diet control or fasting, neither option is a practical solution in a hospital setting for induction of rapid ketosis. While a variety of artificial exogenous ketone supplementation has been known and tried in a variety of forms over the past fifty years, both the chemically created ‘ketone salt’ and synthetic ‘ketone ester’ are non-natural solutions with direct limitations and sub-optimal consequences when deployed in a hospital setting or when used for patient recovery. Ketone chirality matters, and the often “D+L” ketone salts exhibit high salt load coupled with impaired ketosis levels. Moreover, the synthetic ‘ketone ester’ is an alcohol, an artificial, never before seen in man engineered construction of organic chemistry which requires subsequent stomach and liver processing before becoming bioavailable. Consequently, there has been a long felt need and compelling reason to utilize bioidentical D-BetaHydroxyButyric Acid for sepsis mitigation and hospital recovery scenarios. Applicant's exogenous D-Beta HydroxyButyric Acid administered in the absence of a chemical salt or ester and the subject of ClinicalTrials.gov identifier NCT05584371. This is the first time the ‘bioidentical’ form of D-Beta HydroxyButyric Acid in the absence of a ‘ketone salt’ or ‘ketone ester’ has been tested in a hospital setting with positive results.

Bioidentical D-BetaHydroxyButyric Acid creates direct cardiac energy & anti-inflammation resulting in improved therapeutic outcomes. In general, bioidentical free D-Beta HydroxyButyric acid (D-BHB) reduces inflammation, improves atherosclerosis along with improving cardiac energy and reducing glucose or mitigating the glucose spike from carbohydrate consumption. All of these attributes increase opportunities for improved hospitalization outcomes alone or when combined with dietary carbohydrate restriction. Bioidentical free D-BetaHydroxyButyric acid is superior to commonly known ketone salts and ketone esters for a number of reasons, not limited to its rapid bioavailability and absence of chemical salt or ester bond. Free D-BHB acid also has an opportunity for surgical recovery improvement in the absence of co-administered pharmaceuticals as well. D-BHB is direct mitochondrial cardiac, brain and kidney energy & anti-inflammation which synergistically creates enhanced recovery owing to improved mitochondrial bioenergenics during surgical recovery. With enhanced mitochondrial energy, cardiac tissue at risk of dying improve opportunities for recovery for the cardiac cell being challenged, for the cardiac tissue at risk of impairment and for the individual in terms of healthy outcome vs impairment or death.

The ketogenic diet and more recently administration of ketogenic compounds have had a similar long history with proven benefits for conditions such as epilepsy. Dietary interventions for ketogenic diet treatment of atherosclerosis are also yielding compelling results. The administration of the natural form of D-BetaHydroxyButyric Acid as an exogenous compound has rapid onset and direct benefits to cardiac bioenergenics and blood circulation dynamics as well as reduced inflammation including the NLRP3 pathway. Preliminary data indicates that individuals suffering from atherosclerosis often benefit from administration of D-BetaHydroxyButyric Acid in the absence of a salt or ester. The claimed invention utilizes D-BetaHydroxyButyric Acid for hospital recovery intervention as a restorative delivery system for improved physiological outcomes in the treatment of cardiac related physiological conditions among others. The exogenous ketone D-BHB Acid is either co-administered with standard pharmaceutical compounds, prophylactically or subsequently administered with the following expected benefits:

Hospital stay time is reduced and sepsis and septic shock are avoided owing to the anti-inflammatory characteristics of natural D-BetaHydroxyButyric Acid. With the introduction of D-BHB Acid improving cardiac bioenergenics and reducing cardiac inflammation, it is a foreseeable consequence of the claimed invention that a shorter hospital stay after cardiac surgery will be attained leading to a higher percentage of positive physiological recovery breakthroughs.

The bioidentical form of D-BetaHydroxyButyric Acid is rapidly absorbed and immediately bioavailable which is in direct contrast to the moderately ketogenic ketone salts and ketone ester compounds which require stomach disassociation followed by liver processing. The greatly improved bioavailability of free D-BetaHydroxyButyric Acid improves therapeutic outcomes for hospital recovery in conjunction with current standard of care pharmaceuticals as well as when administered alone subsequent to surgical events or even as a prophylactic.

In addition to known glucose reduction attributes, the novel and hereby disclosed prophylactic glucose spike protective effects have substantial protective effects for optimizing metabolic interventions for cancer as well as surgical interventions for cancers including glioblastoma. With glioblastoma surgery in particular, the ability of bioidentical D-BetaHydroxyButyric Acid to both guard against glucose spikes and reduce brain blood glucose in combination with substantial anti-seizure properties will provide substantial benefits in increasing successful outcomes for glioblastoma brain surgery and subsequent ‘push/pull’ anti-cancer metabolic therapies including 6-Diazo-5-oxo-1-norleucine (DON) which is a glutamine antagonist with robust anticancer efficacy and albendazole.

TABLE 1

Free D-β-HydroxyButyric Acid (D-BHB)
Free D-BHB is both mitochondrial energy source
and regulatory compound with the fastest
bioavailability and efficacy vs synthetic ester and
chemical salt.
Unexpected cognitive bioenergenics and
neuroprotection benefits.
Free D-BHB is a rapid release format.
Free D-BHB is safe and non-toxic.
Effects: Regulatory, Direct Mitochondrial Energy
Metabolism: Rapid, minutes
Use in Humans: Ancient
Induces Ketoacidosis: No

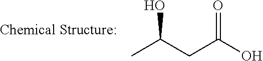

The rationale for a bioidentical D-BHB therapeutic regime is primarily supported by the neuroinflammation reduction properties of bioidentical D-BHB, high ketone levels generated by the same coupled with high bioavailability without salt load or liver metabolism requirements. As the bioidentical ‘free’ form. The bioidentical ‘free’ D-BHB form is immediately bioavailable for rapid metabolism which is materially distinct from the ‘ketone salt’ compounds (often D+L racemic) chemically joined to a salt or the synthetic ‘ketone ester’ compounds which are alcohol based and must be processed in the stomach and liver before releasing the ketone contained therein.

### BRIEF DESCRIPTION OF THE SEVERAL VIEWS OF THE DRAWING(S)

The accompanying drawings are included to better illustrate exemplary embodiments of the claimed invention.

FIG. 1 is a structural illustration of endogenous ketones.

FIG. 2 is a structural illustration of chemical structures of bioavailable free D-BetaHydroxyButyric Acid, ketone ester and ketone salt.

FIG. 3 is a graphical illustration of endogenous ketone levels by age.

FIG. 4 is a graphical illustration of endogenous ketone brain metabolism.

FIG. 5 is a graphical illustration of comparative exogenous ketone blood levels.

FIG. 6 is a graphical illustration of anti-inflammatory metabolism of ketones.

FIG. 7 is a graphical illustration of free D-BetaHydroxyButyric Acid preparation for protection and recovery.

FIG. 8 is a graphical illustration of free D-BetaHydroxyButyric Acid preparation for spinal ischemia recovery.

FIG. 9 is a graphical illustration of D-BHB exogenous ketone blood levels.

FIG. 10 is a graphical illustration of D-BHB exogenous ketone blood levels.

FIG. 11 is a graphical illustration of continuous monitoring of body glucose levels.

FIG. 12 is a graphical illustration of continuous monitoring of body ketone levels resulting in improved deep sleep time due to enhanced D-BHB levels according to the claimed invention.

FIG. 13 is a schematic illustration of a preferred D-BHB sepsis management method with optional sepsis therapeutic pharmaceutical administration.

FIG. 14 is a schematic illustration of a preferred D-BHB sepsis management system with optional sepsis therapeutic pharmaceutical administration.

### DETAILED DESCRIPTION OF THE INVENTION

Introduction: The claimed invention protects against sepsis and enhances hospital recovery by way of two primary embodiments. The first primary embodiment addresses sepsis by reducing inflammation and increasing direct mitochondrial energy by exogenously administering bioidentical D-BetaHydroxyButyric Acid. The second primary embodiment improves existing sepsis therapeutic pharmaceutical interventions by co-administration of D-BetaHydroxyButyric Acid to optimize sepsis therapeutic pharmaceutical compounds.

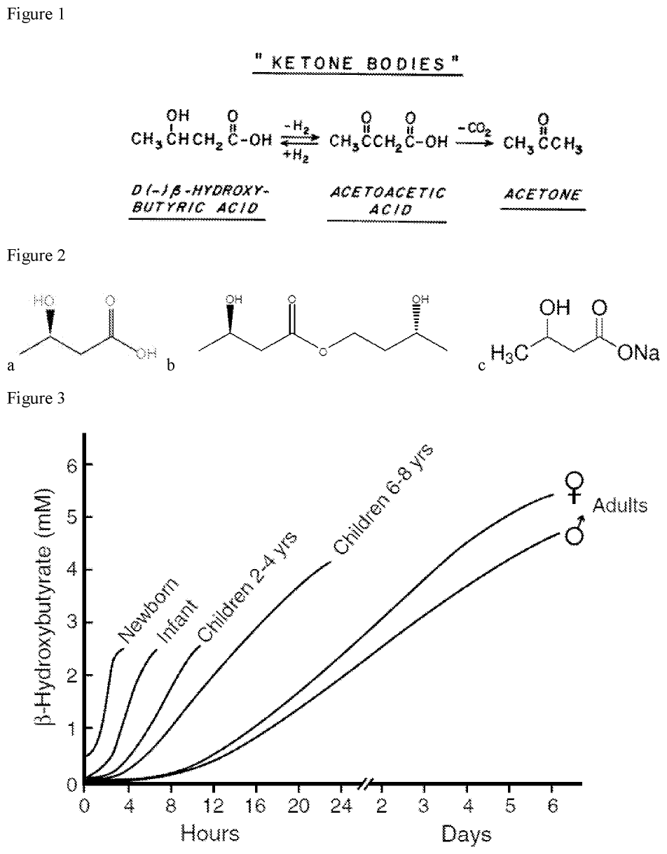

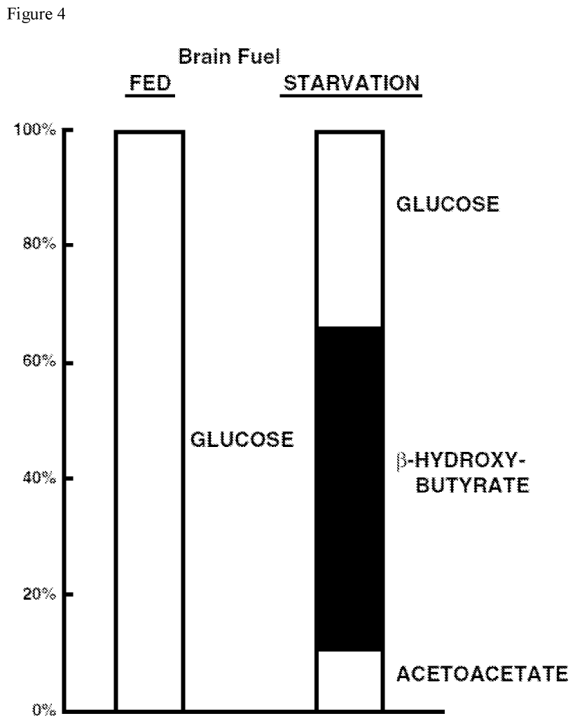

I. D-Beta HydroxyButyric Acid Compound Disclosure and Sepsis applications. FIG. 1 is a structural illustration of endogenous ketones. The illustration of the endogenously produced ketone bodies is from FIG. 4 of the Harvard Medical School Professor/Dr. George F. Cahill, Jr.'s lecture entitled “Ketosis” given over fifty years ago. Dr. Cahill's foundational work provides clear and concise understanding of ketosis to aid in the understanding of the claimed invention. The FIG. 2 (a) chemical formula identifies the claimed bioidentical ‘free’ form of the exogenous D-Beta HydroxyButyric Acid administered in the absence of a chemical salt or ester and is the subject of ClinicalTrials.gov identifier NCT05584371. The claimed exogenously administered bioidentical free D-BetaHydroxyButyric Acid is chemically identical to the D-BetaHydroxyButyric Acid identified in FIG. 1. Greater detail is hereby provided in the submitted BMJ Nutrition, Prevention & Health journal article. To be clear, this publication is the first reported clinical trial of bioidentical free D-BetaHydroxyButyric acid administered exogenously. The intervening fifty years between the Jeremiah Metzger Lecture given in Boston in 1972 and the present clinical trial and disclosure do not detail bioidentical free D-BetaHydroxyButyric Acid but instead disclose a wide variety of precursors, chemical combinations and not naturally occurring synthetic products which are not bioidentical in their administered form.

FIG. 2 is a structural illustration of bioidentical ketone and chemically synthetic chemical ketone structures. FIG. 2(a) depicts bioidentical free BetaHydroxyButyric Acid. FIG. 2(b) depicts the synthetic ketone ester and FIG. 2 (c) shows the chemical ketone salt. Put simply, bioidentical free D-Beta HydroxyButyric Acid offers direct mitochondrial energy resulting in optimized therapeutic outcomes when administered in the absence of a chemical salt or ester configuration. To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long-term concerns. FIG. 2(a) illustration of the chemical form of bioidentical free D-BetaHydroxyButyric Acid is also distinguishable from ketone salts and ketone esters owing to its immediate bioavailability and absence of chemically joined salt or ester bond.

FIG. 3 is a graphical illustration of endogenous ketone levels by age and FIG. 4 is a graphical illustration of endogenous ketone brain metabolism from Dr./Prof Cahill's journal article “Fuel Metabolism in Starvation” provided by Information Disclosure Statement. FIG. 3 details the time to ketosis in healthy adults vs children measured in hours or days. D-BetaHydroxyButyric Acid is a naturally occurring ketone which is produced by the human body. The ketone plays a crucial role during development and throughout the healthy human lifespan. It is produced naturally during fasting (detailed by Cahill's graph above) as well as through the use of ketogenic diets. As Cahill observed, “Due to its use by brain, D-β-HydroxyButyric acid not only has permitted man to survive prolonged starvation, but also may have therapeutic potential owing to its greater efficiency in providing cellular energy in ischemic states such as stroke, myocardial insufficiency, neonatal stress, genetic mitochondrial problems, and physical fatigue.” Cahill's early insights on D-β-HydroxyButyric acid are now through Applicant's clinical trial efforts becoming proven in reality. Applicant's clinical trials including ClinicalTrials.gov identifier NCT05584371 are the first documented clinical applications of free D-BetaHydroxyButyric Acid as further detailed in the provided Dr.Prof. Soto-Mota IDS journal article. FIG. 4 details the fact that ketones in general, and D-BetaHydroxyButyric Acid in particular is the preferred fuel in the human brain. The time to ketosis levels are relevant in the context of the instant application in that creating a rapid 1.5-2.5 mmol/L increase in ketone levels is simply not practical through fasting or diet when rapidly needed in a sepsis protection or hospital recovery context.

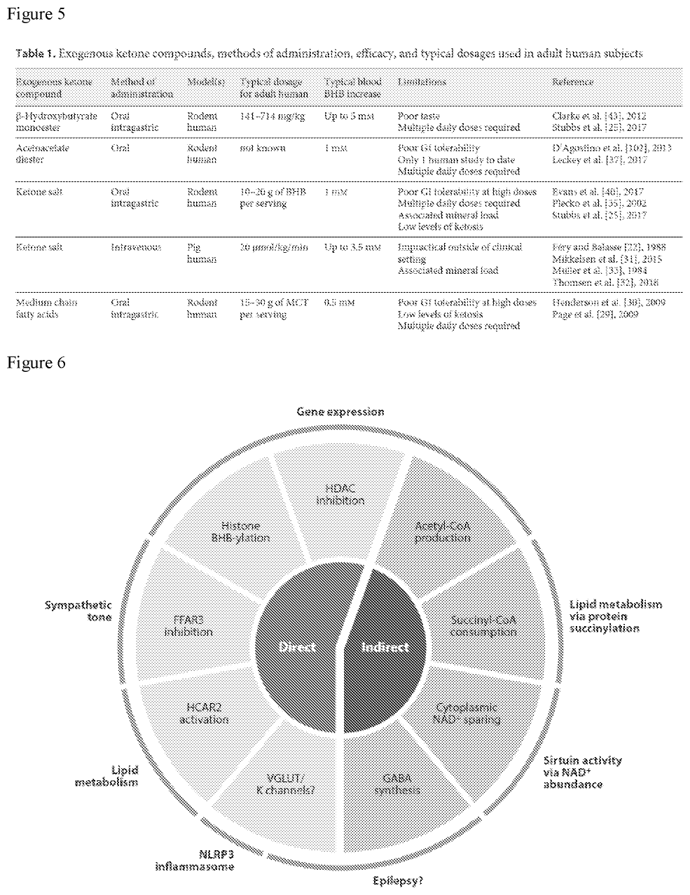

FIG. 5 is a graphical illustration of comparative exogenous ketone blood levels. FIG. 5 depicts Table 1 from “Exogenous Ketone Bodies as Promising Neuroprotective Agents for Developmental Brain Injury” and details the significant limitations among the variety of previously known exogenous salt and ester ketone compounds. Ketone salts are often racemic D+L mixtures which rarely provide greater than 1 mmol/L ketone increase along with substantial salt load rendering it unsuitable for long term daily administration. Similarly, while the synthetic ‘ketone ester’ does provide a higher ketone level response, its highly unpalatable taste renders it highly unsuitable for use in mental health applications. FIG. 5 does not include free D-BetaHydroxyButyric Acid due to the fact that ClinicalTrials.gov identifier NCT05584371 and the provided BMJ Nutrition, Prevention & Health journal article is the first instance of exogenous free D-BetaHydroxyButyric Acid journal publication. Put simply, there has been a longstanding need for a salt free, bioidentical solution to rapid ketosis without alcohol or need for further liver metabolism to rapidly increase ketones to a level relevant for sepsis protection and hospital recovery.

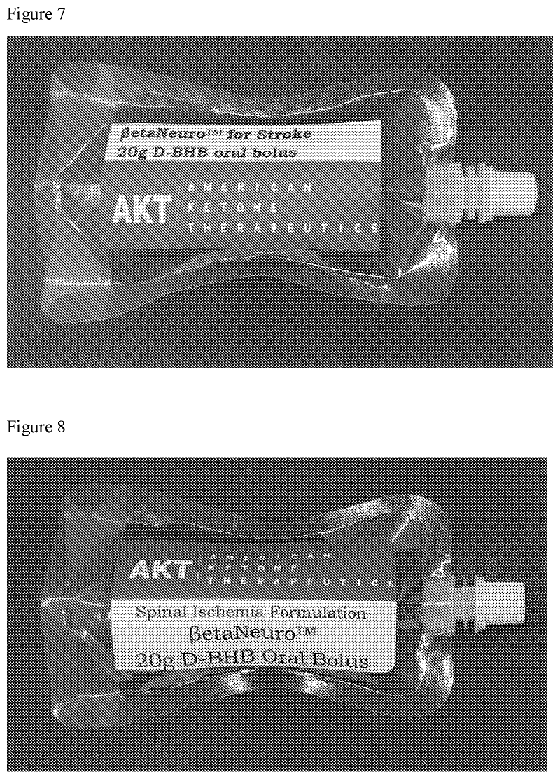

FIG. 6 is a graphical illustration of brain metabolism of ketones from Prof Newman and Prof. Verdin's journal article “β-Hydroxybutyrate:A Signaling Metabolite” figure number 2. This figure details “Schematic of direct and indirect signaling functions of the ketone body BHB. Indirect signaling functions require catabolism to other molecules, whereas direct signaling functions are actions of BHB itself.” Applicants respectfully submits that Professors Newman and Verdin detail the starting point of the anti-inflammatory nature of BetaHydroxyButyric Acid. As supplementation with bioidentical free D-BetaHydroxyButyric Acid is anti-inflammatory it is logical to conclude that reduction of sepsis related events will follow. It is a direct and foreseeable consequence of the claimed invention that similar benefits for recovery from cardiac conditions, stroke and spinal ischemia are achieved. In terms of cardiac applications, Applicant hereby provides as IDS reference 7 the article entitled “Ketone Body β-Hydroxybutyrate Prevents Myocardial Oxidative Stress in Septic Cardiomyopathy” which states “In this study, the ketone body β-OHB was shown to prevent LPS-induced myocardial injury and cardiac dysfunction. Mechanistically, as an HDAC inhibitor, β-OHB activated the antioxidant FoxO3a/MT2 pathway. This response enhanced redox homeostasis and improved mitochondrial function . . . . These findings, therefore, suggest that β-OHB is an effective agent for myocardial protection in sepsis.” (emphasis added) While the study is limited to mouse model applications and utilized the synthetic ketone ester, it is reasonable to conclude that for human applications the bioidentical form of free D-BetaHydroxyButyric Acid will yield superior cardiac protection against sepsis owning to its immediate bioavailability and lack of an alcohol chemical moiety.

Applicant provides by way of IDS5 the article entitled “The ketone metabolite β-hydroxybutyrate blocks NLRP3 inflammasome-mediated inflammatory disease” as similar support for a broader range of anti-inflammatory and consequently reduced sepsis and enhanced hospital recovery applications. The article by Youm states in relevant part on page 6, “These findings suggest that the fasting- or exercise-induced metabolite BHB inhibits the NLRP3 inflammasome in macrophages independently of binding to surface GPR109A receptors or mitochondrial oxidation, which may avoid both competition for receptor occupancy and requirement for ATP generation” and notes that “in states of extreme energy deficit such as starvation, metabolic signals such as BHB can dampen innate immune responses, sparing ATP for the functioning of ketone-dependent organs such as the brain and heart” which holds promise in reducing the “severity of NLRP3-mediated chronic inflammatory diseases.” Consequently, providing bioidentical free D-BetaHydroxyButyric Acid according to the claimed invention should not only reduce incidences of sepsis but also promote enhanced hospital recovery as well.

In terms of sepsis mitigation therapeutic compounds incorporating free D-BetaHydroxyButyric Acid, while preferred embodiments are provided as 5 g-30 g format, larger and smaller amounts may be provided while maintaining the spirit and scope of the invention. In a preferred embodiment, free D-BetaHydroxyButyric Acid is combined and provided in conjunction with a ketogenic complementary nutritional supplement. In an illustrative embodiment, the ketogenic complementary nutritional supplement is selected from arginine, omega-3 fatty acids, and Vitamin D. There is a wide variety of choices for ketogenic complementary supplements which can include precursors such as Medium Chain Triglycerides, plant oils such as olive oil, coconut oil, caprylic acid and other standard ingredients in a ketogenic diet. The primary component to be avoided is any kind of sugar, whether in the form of sucrose, glucose, fructose or the like. Additionally, an alternate preferred embodiment includes a palatability agent incorporating sugar free sweetener such as stevia or allulose as well as natural fruit essence distillate as a palatability enhancer. In an alternate preferred embodiment, one or more sepsis therapeutic pharmaceuticals are added including antibiotics, vasopressors, anticoagulants, corticosteroids and immunoglobulins administered in a physiologically therapeutic amount. FIG. 7 is a graphical depiction of an illustrative example of free D-BetaHydroxyButyric Acid preparation for hospital recovery. The depicted preparation of D-BHB free acid is designed for rapid and easy administration for hospital recovery and may alternately be utilized for sepsis protection. While 20 g D-BHB free acid is depicted a wide range of D-BHB administration is anticipated according to the claimed invention. FIG. 8 is a graphical illustration of free D-BetaHydroxyButyric Acid preparation for spinal ischemia recovery. The depicted preparation of D-BHB free acid is designed for rapid and easy intubated administration. While 20 g D-BHB free acid is depicted a wide range of D-BHB administration in grams is anticipated according to the claimed invention. In addition to the aforementioned bioenergetic properties, long term administration of D-BHB free acid improves spinal ischemia prognosis owing to the enhanced atherosclerosis attributes even in the absence of a co-administered pharmaceutical. It is an anticipated embodiment of the claimed invention that in addition to exigent D-BHB administration, follow-on D-BHB administration between 5 g and 30 g regularly administered over days, weeks, months and even years can improve brain stroke and spinal ischemia positive outcomes and enhance recovery.

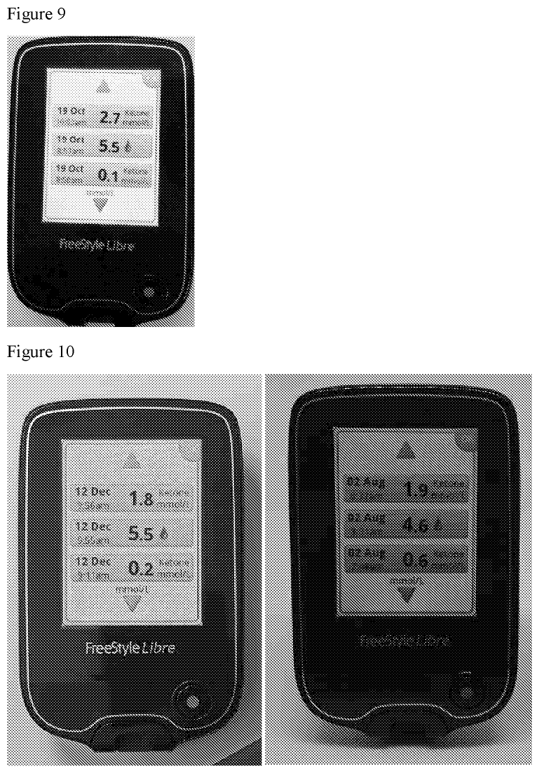

FIG. 9 is a graphical illustration of D-BHB exogenous ketone blood levels. The above Abbot blood ketone meter evidenced by Abbott blood test strip depicts the rapid rise in blood ketone levels from 0.1 mmol/L (absence of ketosis) to 2.7 mmol/L (ketosis) after oral bolus administration of 15 grams of bioidentical free D-BetaHydroxyButyric Acid. The ketone levels depicted are attained in the absence of a salt as well as without ester bonds. It is the free acid D-BHB exogenously administered which is bio-identical to the endogenous D-BHB free acid generated by the human body. The bioidentical ketone body D-BHB is first and foremost an energy source, which is directly and preferentially metabolized by the brain. D-BHB is also active as a signaling mechanism and neuro-inflammation reduction agent, resulting in broad and beneficial brain bioenergy related benefits.

FIG. 10 is a graphical illustration of D-BHB exogenous ketone blood levels. FIG. 10 details rapid ketosis within 45 minutes confirmed by Abbott blood test strip after 10 g D-BHB with palatability agent administered by oral drink. Blood ketone levels document rise from 0.2 mmol/L to 1.8 mmol/L and 0.6 mmol/L to 1.9 mmol/L in a variety of test subjects. FIG. 10 reinforces the fact that supplementation by utilizing free D only BetaHydroxyButyric Acid without ketone salt or ketone ester is a reliable, reproduceable method and system for rapid ketone level increase for stroke recovery and maintenance. While amounts of free D only BetaHydroxyButyric Acid taken for stroke effects may vary, it is an anticipated preferred embodiment that levels between 5 and 30 grams of D only BetaHydroxyButyric Acid are taken as needed for optimum recovery. Alternate embodiments may include lower or higher amounts, however, experimental evidence has confirmed optimal administration in this range for a wide variety of other applications.

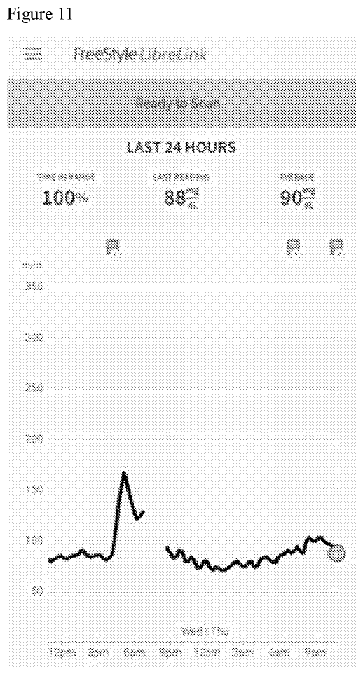

FIG. 11 is a graphical illustration of continuous monitoring of body glucose levels. FIG. 11 depicts continuous glucose monitoring levels with carbohydrate challenge documented by Abbot Libre Continuous Glucose Monitoring (CGM) device. In this illustrative embodiment, glucose levels from a 50 g carbohydrate ingestion are depicted unaided by free D-BetaHydroxyButyric Acid on the left and repeated the following day with two 50 g carbohydrate ingestions accompanied by 10 g free D-BetaHydroxyButyric Acid administered 45 minutes before the carbohydrate insult. The data is noteworthy in that while the equivalent amount of identical carbohydrates were consumed, the rapid glucose spike depicted in the unsupplemented ingestion is not found with 10 g D-BetaHyrdoxyButyric Acid accompaniment. This data is relevant in the stroke context owing to the fact that certain individuals suffering from brain energy deficits often report higher susceptibility due to low ketones and high glucose. As free D-BetaHydroxyButyric Acid can lower glucose while rapidly increasing ketone levels, it is believed that obtaining high ketones while lowering glucose will yield additional stroke recovery benefits for high glucose sensitive individuals suffering from sepsis and may be considered in lieu of insulin administration.

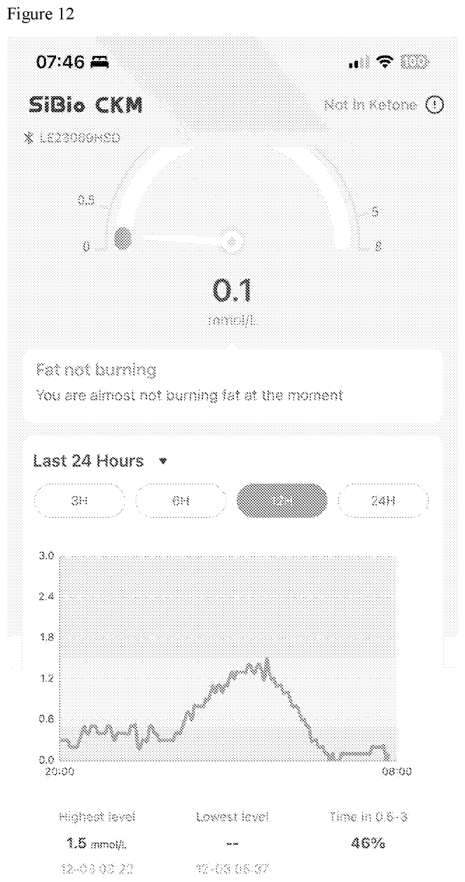

FIG. 12 is a graphical illustration of continuous monitoring of body ketone levels resulting in improved deep sleep time due to enhanced D-BHB levels according to the claimed invention. It is noteworthy that consumption of even small amounts of free D-BetaHydroxyButyric Acid can give rise to therapeutic ketosis levels as measured by Continuous Ketone Meter (CKM). In this representative example, 5 grams of free D-BetaHydroxyButyric Acid consumed 45 minutes prior to bedtime resulted in a 1.5 mmol/L increase can also enhance the quantity of deep sleep obtained. While free D-BetaHydroxyButyric Acid is not a sedative, even small amounts can greatly improve deep sleep quantity and quality. It is an intended and foreseeable embodiment application to increase deep sleep with free D-BetaHydroxyButyric Acid with the aim of guarding against sepsis, improving hospital recovery and enhanced resiliency.

### II. D-Beta HydroxyButyric Acid Sepsis Recovery Method & System Disclosure and Applications for Pharmaceutical Co-Administration

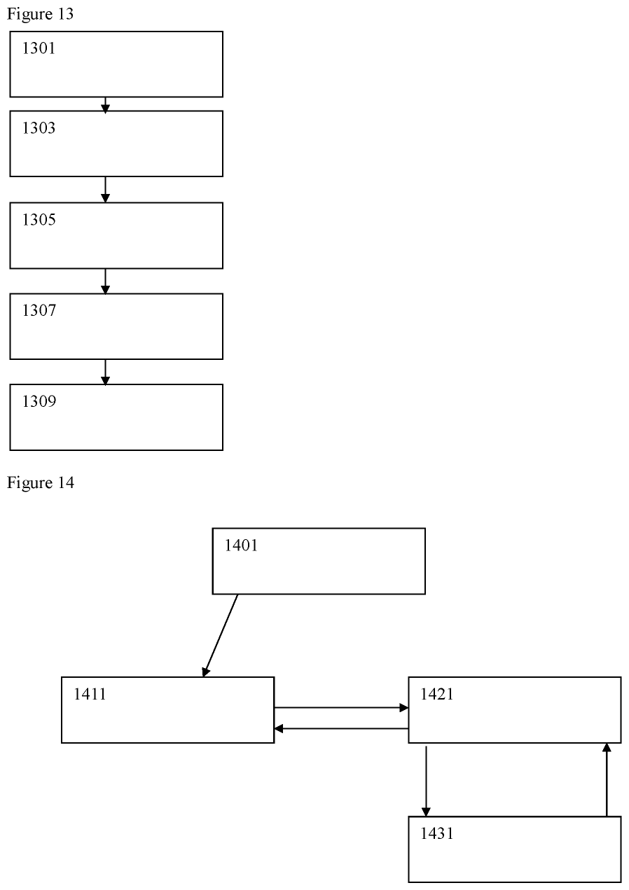

FIG. 13 is a schematic illustration of a preferred bioidentical D-BetaHydroxyButyric Acid sepsis protection/hospital recovery management method with optional pharmaceutical administration. In a preferred embodiment, a method of sepsis symptom management is obtained through the steps of:
Assessing (1301) ketone levels and sepsis symptoms,
Administering (1303) a therapeutic amount of free D-BetaHydroxyButyric acid,
Optionally dosing (1305) a therapeutic amount of sepsis therapeutic pharmaceutical,
Evaluating (1307) the sepsis symptoms of a subject in need, and
Readministering (1309) free D-BetaHydroxyButyric acid alone or in combination with optionally redosing together with a therapeutic amount of sepsis therapeutic pharmaceutical.

In terms of sepsis therapeutic pharmaceuticals, known approaches to treat sepsis include antibiotics, vasopressors, intravenous (IV) fluids, anticoagulants, corticosteroids, insulin and immunoglobulins. Broad-spectrum antibiotics are often the first line of treatment, administered as soon as sepsis is suspected. Once the specific bacteria causing the infection is identified, more targeted antibiotics are used. Vasopressors are used to raise blood pressure in septic shock, a severe condition associated with sepsis where blood pressure drops dangerously low. Common vasopressors include norepinephrine, dopamine, and vasopressin. Intravenous (IV) Fluids are used for fluid resuscitation which is crucial to maintain adequate blood pressure and organ perfusion of which saline or lactated Ringer's solution are commonly used. Anticoagulants are used to prevent or treat disseminated intravascular coagulation (DIC), a complication of sepsis. Anticoagulants include Heparin which is a commonly used anticoagulant for sepsis. Corticosteroids are used in severe cases of sepsis or septic shock, especially when the response to IV fluids and vasopressors is inadequate to reduce inflammation. Insulin Therapy is used to control high blood sugar levels, which can accompany severe sepsis or septic shock. Immunoglobulins may also be used in severe sepsis cases.

FIG. 14 is a schematic illustration of a preferred D-BHB sepsis management system with optional sepsis therapeutic pharmaceutical administration. In the illustrative embodiment, ketone level monitor (1421) determines the amount of free D-BetaHydroxyButyric Acid for a sepsis subject in need thereof, optionally and additionally informed by glucose monitor (1433). Free D-BetaHydroxyButyric Acid therapeutic dose (1411) is administered alone or in conjunction with optional sepsis therapeutic pharmaceutical therapeutic dose (1401).

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments. In particular, free D-BetaHydroxyButyric Acid may be administered diluted or with a preferred palatability agent without detracting from the spirit or scope of the claimed invention.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

I claim:

1. A sepsis protection and enhanced surgical recovery method comprising the steps of:
Assessing ketone levels and sepsis symptoms,
Administering a therapeutic amount of free D-BetaHydroxyButyric acid,
Evaluating the sepsis symptoms of a subject in need, and
Readministering free D-BetaHydroxyButyric acid until sepsis symptoms are mitigated.

2. The method of claim 1 wherein said Administering a therapeutic amount of free D-BetaHydroxyButyric Acid is provided between 5 grams and 30 grams of free D-BetaHydroxyButyric Acid.

3. The method of claim 2 wherein the Assessing ketone levels and sepsis symptoms further comprise assessing bacterial sepsis symptoms.

4. The method of claim 1 additionally comprising dosing a therapeutic amount of sepsis related therapeutic substance selected from the group consisting of antibiotics, vasopressors, intravenous (IV) fluids, anticoagulants, corticosteroids, insulin and immunoglobulins as a co-adjuvant which is simultaneously delivered with Administering a therapeutic amount of Free D-BetaHydroxyButyric Acid.

5. The method of claim 1 additionally comprising dosing a therapeutic amount of sepsis related therapeutic substance as a co-adjuvant delivered more than 15 minutes after Administering a therapeutic amount of Free D-BetaHydroxyButyric Acid.

6. The method of claim 1 additionally comprising dosing a therapeutic amount of sepsis related therapeutic substance as a co-adjuvant delivered more than 15 minutes before Administering a therapeutic amount of Free D-BetaHydroxyButyric Acid.

7. The method of claim 6 additionally comprising Readministering free D-BetaHydroxyButyric Acid.

8. The method of claim 7 wherein the Readministering free D-BetaHydroxyButyric Acid wherein the Readministering free D-BetaHydroxyButyric Acid is performed daily until sepsis symptoms are substantially mitigated.

9. The method of claim 6 wherein the stroke related therapeutic substance is selected from the group consisting of antibiotics, vasopressors, intravenous (IV) fluids, anticoagulants, corticosteroids, insulin and immunoglobulins administered in a physiologically therapeutic amount.

10. The method of claim 9 additionally comprising Readministering free D-BetaHydroxyButyric Acid.

11. A sepsis management system comprising a ketone level monitor which determines the amount of free D-BetaHydroxyButyric Acid for a sepsis subject in need thereof, a glucose monitor and Free D-BetaHydroxyButyric Acid therapeutic dose.

12. The sepsis management system of claim 11 wherein the amount of free D-BetaHydroxyButyric Acid administered is between 5 grams and 30 grams of free D-BetaHydroxyButyric acid.

13. The system of claim 12 wherein the ketone level monitor which determines the amount of free D-BetaHydroxyButyric Acid for a sepsis subject in need thereof additionally comprises assessing bacterial infection symptoms.

14. The sepsis management system of claim 12 additionally comprising a sepsis related therapeutic substance selected from the group consisting of antibiotics, vasopressors, intravenous (IV) fluids, anticoagulants, corticosteroids, insulin and immunoglobulins administered in a physiologically therapeutic amount.

15. The system of claim 11 wherein the ketone level monitor which determines the amount of free D-BetaHydroxyButyric Acid for a sepsis subject in need thereof additionally comprises functionality for daily delivery of free D-BetaHydroxyButyric Acid until sepsis symptoms are substantially mitigated.

16. A sepsis mitigation therapeutic compound comprising free D-BetaHydroxyButyric Acid administered in 5 g-30 g format in conjunction with a ketogenic complementary nutritional supplement.

17. The therapeutic compound of claim 16 wherein said ketogenic complementary nutritional supplement is selected from the group consisting of arginine, omega-3 fatty acids, Medium Chain Triglycerides, olive oil, coconut oil, caprylic acid and Vitamin D.

18. The therapeutic compound of claim 17 wherein said ketogenic complementary nutritional supplement additionally comprises a palatability agent incorporating sugar free sweetener selected from the group consisting of stevia and allulose as well as natural fruit essence distillate as a palatability enhancer.

19. The therapeutic compound of claim 16 additionally comprising one or more sepsis therapeutic pharmaceutical selected from the group consisting of antibiotics, vasopressors, anticoagulants, corticosteroids and immunoglobulins administered in a physiologically therapeutic amount.
